SEQUENCE LISTING (39 sequences)

SEQ ID NO: 1 - RNA, 21 nt, synthetic construct
  source 1..21: mol_type = other RNA
gatctgctcactccggtatct

SEQ ID NO: 2 - RNA, 21 nt, synthetic construct
  source 1..21: mol_type = other RNA
agataccggagtgagcagatc

SEQ ID NO: 3 - RNA, 21 nt, synthetic construct
  source 1..21: mol_type = other RNA
gaccttcctcaagctcaatga

SEQ ID NO: 4 - RNA, 21 nt, synthetic construct
  source 1..21: mol_type = other RNA
agataccggagtgagcagatc

SEQ ID NO: 5 - RNA, 21 nt, synthetic construct
  source 1..21: mol_type = other RNA
ggtttcaaggcaatcgataca

SEQ ID NO: 6 - RNA, 21 nt, synthetic construct
  source 1..21: mol_type = other RNA
agataccggagtgagcagatc

SEQ ID NO: 7 - RNA, 20 nt, synthetic construct
  source 1..20: mol_type = other RNA
aggatatttcagctgccact

SEQ ID NO: 8 - RNA, 20 nt, synthetic construct
  source 1..20: mol_type = other RNA
ccacttcatcttgtccgttg

SEQ ID NO: 9 - RNA, 20 nt, synthetic construct
  source 1..20: mol_type = other RNA
acatgccaaatatcaggatc

SEQ ID NO: 10 - RNA, 25 nt, synthetic construct
  source 1..25: mol_type = other RNA
tcattcctttgttcctatctgtgtg

SEQ ID NO: 11 - protein, 505 aa, Homo sapiens
MKRTPTAEEREREAKKLRLLEELEDTWLPYLTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPRFSSTHRVAECSTGTLDYILQRCQLALQNVCDDVDNDDVSLKSFEPAVLKQGEEIHNEVEFEWLRQFWFQGNRYRKCTDWWCQPMAQLEALWKKMEGVTNAVLHEVKREGLPVEQRNEILTAILASLTARQNLRREWHARCQSRIARTLPADQKPECRPYWEKDDASMPLPFDLTDIVSELRGQLLEAKP

SEQ ID NO: 12 - DNA, 1518 bp, Homo sapiens
  source 1..1518: mol_type = other DNA
atgaagcgcaccccgactgccgaggaacgagagcgcgaagctaagaaactgaggcttcttgaagagcttgaagacacttggctcccttatctgacccccaaagatgatgaattctatcagcagtggcagctgaaatatcctaaactaattctccgagaagccagcagtgtatctgaggagctccataaagaggttcaagaagcctttctcacactgcacaagcatggctgcttatttcgggacctggttaggatccaaggcaaagatctgctcactccggtatctcgcatcctcattggtaatccaggctgcacctacaagtacctgaacaccaggctctttacggtcccctggccagtgaaagggtctaatataaaacacaccgaggctgaaatagccgctgcttgtgagaccttcctcaagctcaatgactacctgcagatagaaaccatccaggctttggaagaacttgctgccaaagagaaggctaatgaggatgctgtgccattgtgtatgtctgcagatttccccagggttgggatgggttcatcctacaacggacaagatgaagtggacattaagagcagagcagcatacaacgtaactttgctgaatttcatggatcctcagaaaatgccatacctgaaagaggaaccttattttggcatggggaaaatggcagtgagctggcatcatgatgaaaatctggtggacaggtcagcggtggcagtgtacagttatagctgtgaaggccctgaagaggaaagtgaggatgactctcatctcgaaggcagggatcctgatatttggcatgttggttttaagatctcatgggacatagagacacctggtttggcgataccccttcaccaaggagactgctatttcatgcttgatgatctcaatgccacccaccaacactgtgttttggccggttcacaacctcggtttagttccacccaccgagtggcagagtgctcaacaggaaccttggattatattttacaacgctgtcagttggctctgcagaatgtctgtgacgatgtggacaatgatgatgtctctttgaaatcctttgagcctgcagttttgaaacaaggagaagaaattcataatgaggtcgagtttgagtggctgaggcagttttggtttcaaggcaatcgatacagaaagtgcactgactggtggtgtcaacccatggctcaactggaagcactgtggaagaagatggagggtgtgacaaatgctgtgcttcatgaagttaaaagagaggggctccccgtggaacaaaggaatgaaatcttgactgccatccttgcctcgctcactgcacgccagaacctgaggagagaatggcatgccaggtgccagtcacgaattgcccgaacattacctgctgatcagaagccagaatgtcggccatactgggaaaaggatgatgcttcgatgcctctgccgtttgacctcacagacatcgtttcagaactcagaggtcagcttctggaagcaaaaccctag

SEQ ID NO: 13 - skipped

SEQ ID NO: 14 - DNA, 21 bp, synthetic construct
  source 1..21: mol_type = other DNA
tgctccaaactcaaaacggaa

SEQ ID NO: 15 - DNA, 23 bp, synthetic construct
  source 1..23: mol_type = other DNA
atcattatggggatttaagctct

SEQ ID NO: 16 - DNA, 21 bp, synthetic construct
  source 1..21: mol_type = other DNA
attgttgaaaagtttcgctct

SEQ ID NO: 17 - DNA, 21 bp, synthetic construct
  source 1..21: mol_type = other DNA
gaacatatttgcaggtgtcca

SEQ ID NO: 18 - DNA, 21 bp, synthetic construct
  source 1..21: mol_type = other DNA
agcactctaagcctcaacaca

SEQ ID NO: 19 - RNA, 22 nt, synthetic construct
  source 1..22: mol_type = other RNA
atttaaaaggcaaccggagcat

SEQ ID NO: 20 - DNA, 21 bp, synthetic construct
  source 1..21: mol_type = other DNA
acagcctcggtttagttccac

SEQ ID NO: 21 - DNA, 21 bp, synthetic construct
  source 1..21: mol_type = other DNA
tgcctcagccactcaaactcc

SEQ ID NO: 22 - DNA, 22 bp, synthetic construct
  source 1..22: mol_type = other DNA
acaatcacgtcatagctaccag

SEQ ID NO: 23 - DNA, 20 bp, synthetic construct
  source 1..20: mol_type = other DNA
tgagaccacaccttcacgtt

SEQ ID NO: 24 - DNA, 22 bp, synthetic construct
  source 1..22: mol_type = other DNA
cgcggtcatcaacgactaccag

SEQ ID NO: 25 - DNA, 21 bp, synthetic construct
  source 1..21: mol_type = other DNA
aaagcacaggttccgacaccc

SEQ ID NO: 26 - DNA, 22 bp, synthetic construct
  source 1..22: mol_type = other DNA
cacgacagaaggagagcagaag

SEQ ID NO: 27 - DNA, 20 bp, synthetic construct
  source 1..20: mol_type = other DNA
gctactgccgtccgattgag

SEQ ID NO: 28 - DNA, 20 bp, synthetic construct
  source 1..20: mol_type = other DNA
gaagagcgacccacacgtca

SEQ ID NO: 29 - DNA, 21 bp, synthetic construct
  source 1..21: mol_type = other DNA
ccagttcgtttcagtgccaca

SEQ ID NO: 30 - DNA, 21 bp, synthetic construct
  source 1..21: mol_type = other DNA
ccttcctgatggccttacgtg

SEQ ID NO: 31 - DNA, 19 bp, synthetic construct
  source 1..19: mol_type = other DNA
aaaataaccgtctcgctgt

SEQ ID NO: 32 - DNA, 21 bp, synthetic construct
  source 1..21: mol_type = other DNA
ctagaacctgccctgattccc

SEQ ID NO: 33 - DNA, 21 bp, synthetic construct
  source 1..21: mol_type = other DNA
aacccagacttgtatcggtca

SEQ ID NO: 34 - DNA, 20 bp, synthetic construct
  source 1..20: mol_type = other DNA
caggttgtctcctgcgactt

SEQ ID NO: 35 - DNA, 20 bp, synthetic construct
  source 1..20: mol_type = other DNA
tatgggggtctgggatggaa

SEQ ID NO: 36 - DNA, 19 bp, synthetic construct
  source 1..19: mol_type = other DNA
ttcgtattgcgccgctaga

SEQ ID NO: 37 - DNA, 20 bp, synthetic construct
  source 1..20: mol_type = other DNA
ctttcgctctggtccgtctt

SEQ ID NO: 38 - RNA, 19 nt, synthetic construct
  source 1..19: mol_type = other RNA
gaggatccaaggcaaagat

SEQ ID NO: 39 - RNA, 19 nt, synthetic construct
  source 1..19: mol_type = other RNA
taaggctatgaagagattt

(19) United States
(12) Patent Application Publication
(10) Pub. No.: US 2026/0174858 A1
(43) Pub. Date: Jun. 25, 2026

(54) TARGETING THE m6A MRNA DEMETHYLASE FTO

(71) Applicants: Jayakrishna AMBATI, Charlottesville, VA (US); Shao-bin WANG, Charlottesville, VA (US); University of Virginia Patent Foundation, Charlottesville, VA (US); UNIVERSITY OF VIRGINIA, Charlottesville, VA (US)

(72) Inventors: Jayakrishna Ambati, Charlottesville, VA (US); Shao-bin Wang, Charlottesville, VA (US)

(21) Appl. No.: 19/124,706

(22) PCT Filed: Oct. 27, 2023
(86) PCT No.: PCT/US2023/078045

§ 371 Date: Apr. 25, 2025

Related U.S. Application Data
(60) Provisional application No. 63/381,163, filed on Oct. 27, 2022.

(51) Int. Cl. A61P 35/00

(52) U.S. Cl. CPC A61K 45/06; A61P 35/00

(57) ABSTRACT

Provided herein are methods to treat fibrosis and/or inhibit angiogenesis.

### PRIORITY

This application claims priority to and the benefit of U.S. Provisional Patent Application No. 63/381,163, filed Oct. 27, 2022, the entire disclosures of which are incorporated herein by this reference.

### GOVERNMENT GRANT SUPPORT

This invention was made with government support under Grant Nos. EY028027, EY029799, EY031039 and AG082108 awarded by the National Institutes of Health. The government has certain rights in the invention.

### INCORPORATION BY REFERENCE OF SEQUENCE LISTING

This application contains a sequence listing. It has been submitted electronically as an XML file titled “1036334WO1.xml.” The sequence listing is 36,694 bytes in size and was created on Oct. 27, 2023. It is hereby incorporated by reference in its entirety.

### BACKGROUND OF THE INVENTION

Vascular endothelial growth factor-A (VEGFA, also known as VEGF) is an angiogenic factor that regulates the physiological and pathological blood vessel growth (1). Increased abundance of VEGF in the eye underlies many forms of aberrant ocular angiogenesis and resultant vision loss, including in neovascular age-related macular degeneration (nvAMD), proliferative diabetic retinopathy (PDR), ischemic retinal vein occlusion, and retinopathy of prematurity (ROP) (2). Multiple VEGF inhibitors are approved for such ocular angiogenic diseases. Despite the initial, and often dramatic, efficacy of anti-VEGF therapy, real-world and long-term studies are more sobering (3,4).

### SUMMARY

M6-methyladenosine (m6A) is an endogenous RNA modification that plays a role in many pathological processes including cancer and inflammatory diseases. Provided herein is a novel signaling pathway that responds to the pathophysiological m6A mRNA demethylation, and the use of m6A demethylases-inhibiting compounds, in particular fat mass- and obesity-associated protein (FTO) inhibitors, for the treatment and/or prevention of fibrosis (scarring disorders/diseases) and disorders/disease involving angiogenesis (aberrant blood vessel growth/formation).

One aspect provides a method to treat and/or prevent fibrosis comprising administering to a subject in need thereof an effective amount of at least one M6-methyladenosine (m6A) demethylase-inhibiting compound. One aspect provides a method to treat and/or prevent an angiogenesis in an angiogenesis-dependent disease comprising administering to a subject in need thereof an effective amount of at least one N6-methyladenosine (m6A) demethylase-inhibiting compound. One aspect provides a method to inhibit fibrosis comprising administering to a subject in need thereof an effective amount of at least one N6-methyladenosine (m6A) demethylase-inhibiting compound. One aspect provides a method to inhibit angiogenesis comprising administering to a subject in need thereof an effective amount of at least one N6-methyladenosine (m6A) demethylase-inhibiting compound.

In one aspect, the at least one m6A demethylase-inhibiting compound is a fat mass- and obesity-associated protein (FTO) inhibitor. In aspect the subject is a mammal, such as human.

In one aspect, the fibrosis comprises one or more of acute fibrosis, acute kidney injury, adhesive capsulitis, aerosols, Alcoholic liver disease, alpha-1-antitrypsin deficiency, Amyloidosis, Arrhythmogenic right ventricular cardiomyopathy, arthrofibrosis, asbestosis, asthma, Atrial Fibrosis, Autoimmune diseases, Autoimmune glomerular diseases, Autoimmune hepatitis, burn induced fibrosis, carbon pneumoconiosis, cardiac fibrosis, catheter placement, chemical dusts, chemotherapy/radiation induced pulmonary fibrosis, chronic obstructive pulmonary disease (COPD), chronic renal failure, chronic renal insufficiency, Chronic viral hepatitis, cirrhosis, coal worker's pneumoconiosis, collagenous colitis, complications from pneumoconiosis, conjunctival fibrosis, corneal fibrosis, coronary artery disease, Crohn's disease, cystic fibrosis, deltoid fibrosis, diabetes, Diabetic glomerulosclerosis, drug induced ergotism, drug reaction and exposure to toxins, Drug-induced, drug-induced interstitial lung disease, drug-induced interstitial lung disease, Drug-induced nephrogenic systemic fibrosis, Dupuytren's contracture, emerging cirrhosis, end stage renal disease or renal failure, endomyocardial fibrosis, eosinophilic fasciitis, Familial hypertrophic cardiomyopathy, familial pulmonary fibrosis, fibromyalgia, fibrosis associated with atherosclerosis, fumes or vapors, Gaucher's disease, general fibrosis syndrome characterized by replacement of normal muscle tissue by fibrous tissue, generalized morphea, glomerulonephritis, glomerulosclerosis, pulmonary fibrosis, glycogenosis, Graft versus host disease, hepatitis B virus infection, hepatitis C virus infection, hepatitis D virus infection, Hypersensitive pneumonitis, hypersensitivity pneumonitides, Hypertensive nephrosclerosis, idiopathic pulmonary fibrosis, Infectious myocarditis, Infectious pneumonitis, inflammatory bowel disease, Inherited disorders, Inherited metabolic disorders, interstitial fibrotic lung disease, interstitial lung disease, Intestinal bypass, Keloid, kidney fibrosis including glomerular sclerosis, kidney fibrosis resulting from dialysis, linear scleroderma, liver cirrhosis, liver fibrosis, lymph node fibrosis, Macular degeneration, mediastinal fibrosis, multifocal fibrosclerosis, myelodysplastic syndrome, myelofibrosis, myeloproferative syndrome, myocardial fibrosis, myocardial infarction, NASH associated cirrhosis obesity, nephrogenic systemic fibrosis, nephropathy, nodular fascilitis, non-alcoholic steatohepatitis (NASH), non-cirrhotic hepatic fibrosis, Nonalcoholic fatty liver disease, oral fibrosis, organ specific fibrosis, pancreatitis, peritoneal fibrosis, Peyronie's disease, Pneumoconiosis, Post-radiation, Pressure-overload heart, primary biliary cirrhosis, progressive kidney disease, Progressive massive fibrosis, progressive renal disease or diabetic nephropathy, protein malnutrition, pulmonary fibrosis, pulmonary fibrosis caused by an infectious agent, pulmonary fibrosis caused by inhalation of inorganic dust, pulmonary fibrosis caused by inhalation of noxious gases, pulmonary hypertension, radiation induced fibrosis, renal fibrosis, renal tubulointerstitial fibrosis, retinal fibrosis, retroperitoneal fibrosis, Sarcoidosis, scarring fibrosis, Schistosomiasis, Scleroderma including morphea, scleroderma/systemic sclerosis, sclerodermatous graft-vs-host-disease, Secondary amyloidosis, silicosis, Storage disorders, e.g. hemochromatosis, Subretinal fibrosis, systemic sclerosis, Toxic environmental exposure, Transplant rejection, trauma induced fibrosis, Tuberculosis, valvular disease, viral induced hepatic cirrhosis, wound healing fibrosis or combinations thereof.

In one aspect, the angiogenesis occurs in an angiogenesis dependent disease or disorder comprising one more ore cancer, diabetic retinopathy, autoimmune diseases, rheumatoid arthritis, atherosclerosis, cerebral ischemia, neovascular macular degeneration, corneal neovascularization, iris neovascularization, subretinal neovascularization, choroidal neovascularization, cardiovascular diseases or delayed wound healing. In one aspect, the cancer comprises one or more of lung cancer, adenocarcinoma, adenocarcinoma of the lung, squamous carcinoma, squamous carcinoma of the lung, malignant mixed mullerian tumor, head and/or neck cancer, breast cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, stomach cancer, prostate cancer, testicular cancer, ovarian cancer, cervical cancer, endometrial cancer, uterine cancer, pancreatic cancer, colon cancer, rectal cancer, colorectal, gastric cancer, kidney cancer, bladder cancer, spleen cancer, thymus cancer, thyroid cancer, brain cancer, neuronal cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, neuroblastoma, white blood cell cancer (e.g., lymphoma, leukemia, etc.), hereditary non-polyposis cancer (HNPC), and/or colitis-associated cancer.

In one aspect, the inhibitor comprises an inhibitory nucleic acid molecule. In one aspect, the inhibitory nucleic acid molecule comprises an antisense nucleic acid molecule, a small interfering RNA (siRNA), or a short hairpin RNA (shRNA). In one aspect, the inhibitory nucleic acid molecule comprises one or more of 5′-P-GAU CUG CUC ACU CCG GUA UCU-3′ (SEQ ID NO: 1), 5′-P-AGA UAC CGG AGU GAG CAG AUC-3′ (SEQ ID NO: 2), 5′-P-GAC CUU CCU CAA GCU CAA UGA-3′ (SEQ ID NO: 3), 5′-P-AGA UAC CGG AGU GAG CAG AUC-3′ (SEQ ID NO: 4), 5′-P-GGU UUC AAG GCA AUC GAU ACA-3′ (SEQ ID NO: 5), 5′-P-AGA UAC CGG AGU GAG CAG AUC-3′ (SEQ ID NO: 6), 5′-mAmGmGmAmUATTTCAGCTGmCmCmAmCmU-3′ (SEQ ID NO: 7), 5′-mCmCmAmCmUTCATCTTGTCmCmGmUmUmG-3′ (SEQ ID NO: 8), 5′-mAmCmAmUmGCCAAATATCAmGmGmAmUmC-3′ (SEQ ID NO: 9), and/or 5′-mTmCmAmUmUCCTTTGTTCCmAmCmGmGmG-3′ (SEQ ID NO: 10).

In one aspect, the inhibitor comprises a small molecule. In one aspect, the small molecule comprises one or more of

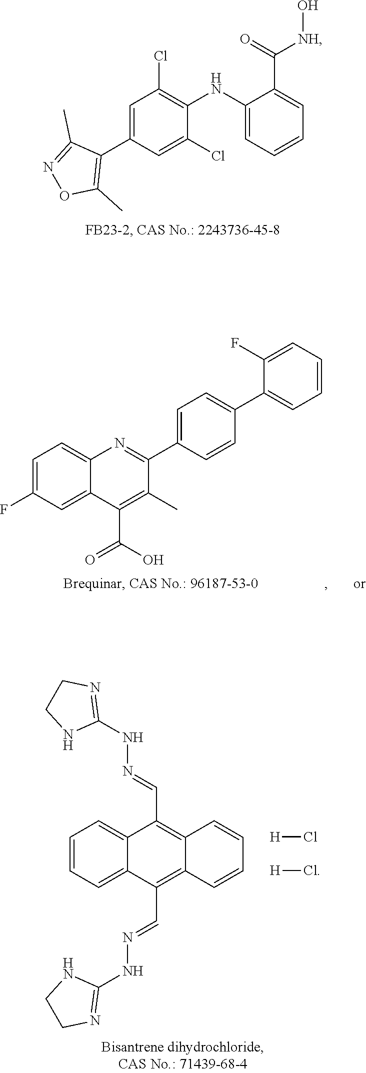

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1C. Bacterial lipopolysaccharide (LPS) downregulates m6A methylome components (A). The RNA-seq analysis of primary BMDCs treated with LPS for 3 or 6 hours. Values represent as Peak Over Input (POI) scores. (B) Wild type of bone marrow-derived macrophages (BMDM) were treated with 250 ng/ml of LPS (Escherichia coli O111:B4) for 6 hours. Total RNAs were extracted by Trizol reagent and quantified with Nanodrop. The mRNA purification, library perpetration and RNA-seq was performed on a BGI-SEQ 500 platform and analyzed by Beijing Genomics institution (BGI). (C). Heatmap graph show the expression levels of the components of RNA m6A methylome based on the dataset from RNA-seq. Results show LPS exposure generally downregulates the expression levels of RNA m6A methylome components.

FIGS. 2A-2B. Toll-like receptors (TLR)/Myd88 signaling axis is required for LPS-induced m6A methylome suppression. (A). Wild type BMDMs were treated with 250 ng/ml of LPS (Escherichia coli O111:B4) for 6 hours. Total RNAs were extracted by Trizol reagent and quantified with Nanodrop. cDNA was synthesized using a QuantiTect Reverse Transcription kit (QIAGEN). The abundance of METTL3 and METTL14 mRNA were quantified by real-time quantitative PCR (Applied Biosystems 7900 HT Fast Real-Time PCR system) with Power SYBR green Master Mix. (B). Wild type (WT), Tlr 23479−/− (Tlr−/−) and Myd88−/− BMDMs were treated with 250 ng/ml of LPS (Escherichia coli O111:B4) for 6 hours. The abundance of METTL3 mRNA waw quantified by real-time qPCR. Values represent as the means±SE, n=3, **p<0.01; ns, not significant. two-way ANOVA with Sidak's multiple comparisons. Results show LPS-induced downregulations of METTL3 and METTL14 were restored in Tlr−/− and Myd88−/− BMDMs.

FIGS. 3A-3B. LPS exposure enhances m6a demethylation depends on Myd88 signaling. (A, B). Wild type- or Myd88−/− BMDMs were treated with 250 ng/ml of LPS (Escherichia coli 0111:B4) for indicated time points. Total RNAs were extracted by Trizol reagent and quantified with Nanodrop. Global RNA m6A methylation were measured by m6A dot-blotting. Methylene blue (MB) was used as loading control.

FIG. 4. LPS exposure reduces core complexes of m6A methylome via Myd88 signaling. Human monocytes (THP-1) were pre-treated MYD88 inhibitory peptides (MYD88i) or TBK1 inhibitor (TBK1i) for 1 hour, followed with 250 ng/ml of LPS (Escherichia coli O111:B4) for 6 hours. Cell lysates were collected and subjected to SDS-PAGE. Immunoblots of indicated proteins were used to evaluate the expression levels of RNA m6A methylome components. Results show that LPS treatment significantly reduced the protein levels of VIRMA, METTL3 and METTL14, which were impaired by MYD88 or TBK1 inhibition.

FIGS. 5A-5B. LPS exposure increase the m6A demethylase, FTO (A). Wild type BMDMs were treated with 250 ng/ml of LPS (Escherichia coli O111:B4) for 6 hours. Cell lysates were collected subjected to immunoblotting, for evaluating the expression levels of m6A demethylase, FTO. Actin was used as loading control. (B). Quantification of global m6A RNA in DMSO (Vehicle) or FTO inhibitor (FTOi) pretreated wild-type BMDM followed with LPS exposure for 6 hours. Values represent as the means±SE, n=3, **p<0.01; *p<0.05. two-way ANOVA with Sidak's multiple comparisons.

FIG. 6. FTO inhibition reduces LPS-induced IL-6 release on BMDMs. Wild type BMDMs were pre-treated with various dosage of FTO inhibitor (FB23-2, 20 uM) 1 hour, followed with 250 ng/ml of LPS (Escherichia coli O111:B4) for 6 hours. Measurement of IL-6 release by ELISA assay. Values represent as the means±SE, n=3, ****p<0.0001. one-way ANOVA with Dunnett's multiple comparisons test.

FIG. 7. FTO inhibition rescues the mice death in the LPS model of septic shock. Wild type C57BL/6J mice were pretreated with FTOi (20 mg/kg) for 16 hours via intraperitoneal injection. Next day, the mice were challenged with 10 mg/kg LPS. Survival curves were analyzed by log-rank (Mantel-Cox) test in DMSO (Vehicle) or FTOi treated mice. *p<0.05. n=13 (Vehicle), n=14 (FTOi).

FIGS. 8A-8C. FTO inhibitor treatment impairs serum cytokines release in the LPS model of sepsis (A, B, C). Wild type C57BL/6J mice were pretreated with FTOi (20 mg/kg) for 16 hours via intraperitoneal injection. Next day, the mice were challenged with 10 mg/kg LPS. Mice serums were collected at 10 hours after LPS administration. Measurement of IL-6 (m), TNF-α (n) and IL-1α levels with serum collected from non-simulated (NT), Vehicle+LPS and FTOi+LPS treated mice by ELISA. Values represent as the means±SE, n=3, *p<0.05; **p<0.01; ***p<0.001. one-way ANOVA with Dunnett's multiple comparisons test.

FIGS. 9A-9C. FTO upregulated in the laser-induced Choroidal Neovascularization (CNV) A, B, C). Choroidal Neovascularization was induced with Wild type mice. At 3 days after laser injury, eyes were enucleated, and total RNA extracted from retina and RPE-choroid complexes. The expression of Fto and Alkbh5 in control and CNV tissues were determined by qPCR.

FIGS. 10A-10C. FTO inhibitor treatment inhibits angiogenesis in the laser-induced Choroidal Neovascularization (CNV) (A, B, C). Wild type C57BL/6J mice were pretreated with FTOi (20 mg/kg) for 16 hours via intraperitoneal injection. Next day, each group of mice injected with 25 ng FTOi or equal amount of DMSO into the vitreous humor immediately after laser injury. At 7 days after the laser injury, eyes were enucleated and CNV volumes were determined by FITC-lectin B4 staining. Macrophage visualized by F4/80 staining.

FIGS. 11A-11B. FTO inhibitor inhibits VEGFA release in mouse BMDMs (A, B). Wild type BMDMs treated with FTOi (20 uM) or DMSO for 12 hours, then following with or without LPS (250 ng/ml) stimulation for 6 hours. Supernatants were collected for VEGFA ELISA assay. Values represent as the means±SE, n=3, *p<0.05; ***p<0.001; ****p<0.0001. two-way ANOVA with Sidak's multiple comparisons.

FIGS. 12A-12B. FTO inhibitor inhibits VEGFA release in human THP-1 cells. (A, B). Human THP-1 cells were treated with indicted dosage of FTOi or DMSO for 24 hours or 48 hours. Supernatants were collected for VEGFA ELISA assay. Values represent as the means±SE, n=3, *p<0.05; **p<0.01; ***p<0.001. two-way ANOVA with Sidak's multiple comparisons.

FIGS. 13A-13B. FTO inhibitor inhibits VEGFA release in vivo. (A, B). Wild type C57BL/6J mice were pretreated with FTOi (20 mg/kg) for 16 hours via intraperitoneal injection. Next day, each group of mice injected with 25 ng FTOi or equal amount of DMSO into the vitreous humor immediately after laser injury. At 3 days after the laser injury, eyes were enucleated and subjected to VEGF-A ELISA Assay. Values represent as the means±SE, n=3, *p<0.05; one-way ANOVA with Dunnett's multiple comparisons test.

FIGS. 14A-14B. FTO is required for VEGFA release in mouse BMDMs. (A, B). BMDMs were transfected with siRNA targeting control or Fto sequence. The knockdown efficacy was measured by qPCR, and VEGFA release was determined by ELISA. Values represent as the means±SE, n=3, ns, no significant; ***p<0.001; ****p<0.0001. two-way ANOVA with Sidak's multiple comparisons.

FIGS. 15A-15B. FTO inhibition increase the level of methylated (m6A) VEGFA mRNA. (A, B). BMDMs were pretreated with FTOi or DMSO for 16 hours. Next day, the cells were collected and the level of methylated VEGFA mRNA was measured by MeRIP and qPCR. Values represent as the means±SE, n=3, ns, no significant; **p<0.01; ***p<0.001. two-way ANOVA with Sidak's multiple comparisons.

FIGS. 16A-16B. FTO upregulated in fibrotic lesion of advanced stage of CNV. (A, B) Choroidal Neovascularization was induced with Wild type mice. At 7 and 21 days after laser injury, eyes were enucleated, and RPE-choroid complexes were collected. The expression of Fto and subretinal fibrosis associated genes in control and CNV tissues were determined by qPCR. The expression levels of FTO and Collagen type 1, the subretinal fibrosis marker were illustrated by immunofluorescence staining. Griffonia Simplicifolia Lectin I (GSL I) Isolectin B4 used to determine the neovascularization.

FIGS. 17A-17B. FTO inhibition suppress the fibrotic response in human RPE cell (A) Human RPE cell line, ARPE-19 was pretreated with FTOi or DMSO at indicated concentrations, and then stimulated with recombinant human TGF-β1 (10 ng/ml) for 12 hours. The expression levels of fibrotic markers, Alpha-Smooth Muscle Actin (αSMA), fibronectin (FN), and FTO, actin was determined by immunoblotting. (B) Immunofluorescence staining of fibronectin (FN) in ARPE-19 cells stimulated with recombinant human TGF-β1 (10 ng/ml) and treated with FTOi or DMSO.

FIGS. 18A-18B. Exogenous expression of FTO induces fibrosis response in human RPE cell. (A, B) Human RPE cell line, ARPE-19 was transduced with lentivirus expressing GFP control or human FTO proteins. The expression levels of FTO and fibrosis-related genes, ACTA2 (encoding αSMA), FN1 (encoding fibronectin), COL1A1 (Collagen type 1), CCN2 (Connective Tissue Growth Factor, CTGF), and TGF-β receptors (1, 2, 3, LRP1) were determined by q-PCR.

### DESCRIPTION OF THE INVENTION

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of 5%, 10%, +20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

With regard to administering the compound, the term “administering” refers to any method of providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intra-aural administration, intracerebral administration, rectal administration, and parenteral administration, including injectable such as intravenous administration, intra-arterial administration, intramuscular administration, subcutaneous administration, intra vitreous administration, including via intravitreous sustained drug delivery device, intracameral (into anterior chamber) administration, suprachoroidal injection, subretinal administration, subconjunctival injection, sub-Tenon's administration, peribulbar administration, transscleral drug delivery, administration via topical eye drops, and the like. Administration can be continuous or intermittent. In various aspects, a preparation can be administered therapeutically; that is, administered to treat an existing disease or condition (e.g., exposure to OP compounds). In further various aspects, a preparation can be administered prophylactically; that is, administered for prevention of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition. For example, for oral administration a dose or doses of up to about 1200 mg per day or up to about 600 mg per day or up to about 300 mg per day or up to about 150 mg per day or up to about 50 mg per day can be administered. For example, for intra-vitreous administration a dose or doses of up to about 1 mg or up to about 0.5 mg or up to about 0.25 mg or up to 0.1 about mg can be administered.

The terms “treating,” “treat” and “treatment” include (i) preventing a disease, such as atherosclerosis, plaque buildup, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit,” “inhibiting,” and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting. As will be recognized by one of ordinary skill in the art, the terms “suppression,” “suppressing,” “suppressor,” “inhibition,” “inhibiting” or “inhibitor” do not refer to a complete elimination in all cases. Rather, the skilled artisan will understand that the term “suppressing” or “inhibiting” refers to a reduction or decrease. Such reduction or decrease can be determined relative to a control. In some embodiments, the reduction or decrease relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

The terms “subject” or “subject in need thereof refer to a target of administration, which optionally displays symptoms related to a particular disease, condition, disorder, or the like. The subject(s) of the herein disclosed methods can be human or non-human (e.g., primate, horse, pig, rabbit, dog, sheep, goat, cow, cat, guinea pig, rodent, and non-mammals). The term “subject” does not denote a particular age or sex. Thus, adult and newborn subjects, as well as fetuses, whether male or female, are intended to be covered. The term “subject” includes human and veterinary subjects.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.” The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As described herein, the presently disclosed subject matter further includes pharmaceutical compositions comprising the compounds described herein together with a pharmaceutically acceptable carrier.

The term “pharmaceutically acceptable carrier” refers to sterile aqueous or nonaqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents {e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycol late); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compositions can be formulated as eye drops. For example, the pharmaceutically acceptable carrier may comprise saline solution or other substances used to formulate eye drop, optionally with other agents. Thus, eye drop formulations permit for topical administration directly to the eye of a subject.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

The presently disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

As used herein, the term “providing a prognosis” refers to providing information regarding the impact of the presence of the disease/disorder (e.g., as determined by the diagnostic methods of the present invention) on a subject's future health.

By the term “specifically binds to,” as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of this invention.

As used herein, a “substantially homologous amino acid sequences” includes those amino acid sequences which have at least about 95% homology, at least about 96% homology, at least about 97% homology, at least about 98% homology, or at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

Provided herein are methods to treat fibrosis disorders, angiogenesis disorders or to inhibit fibrosis and/or angiogenesis. Examples of fibrosis disorders to where methods to inhibit fibrosis are needed, include, but are not limited to, fibrosis or fibrosis due to, such as acute fibrosis, acute kidney injury, adhesive capsulitis, aerosols, Alcoholic liver disease, alpha-1-antitrypsin deficiency, Amyloidosis, Arrhythmogenic right ventricular cardiomyopathy, arthrofibrosis, asbestosis, asthma, Atrial Fibrosis, Autoimmune diseases, Autoimmune glomerular diseases, Autoimmune hepatitis, burn induced fibrosis, carbon pneumoconiosis, cardiac fibrosis, catheter placement, chemical dusts, chemotherapy/radiation induced pulmonary fibrosis, chronic obstructive pulmonary disease (COPD), chronic renal failure, chronic renal insufficiency, Chronic viral hepatitis, cirrhosis, coal worker's pneumoconiosis, collagenous colitis, complications from pneumoconiosis, conjunctival fibrosis, corneal fibrosis, coronary artery disease, Crohn's disease, cystic fibrosis, deltoid fibrosis, diabetes, Diabetic glomerulosclerosis, drug induced ergotism, drug reaction and exposure to toxins, Drug-induced, drug-induced interstitial lung disease, drug-induced interstitial lung disease, Drug-induced nephrogenic systemic fibrosis, Dupuytren's contracture, emerging cirrhosis, end stage renal disease or renal failure, endomyocardial fibrosis, eosinophilic fasciitis, Familial hypertrophic cardiomyopathy, familial pulmonary fibrosis, fibromyalgia, fibrosis associated with atherosclerosis, fumes or vapors, Gaucher's disease, general fibrosis syndrome characterized by replacement of normal muscle tissue by fibrous tissue, generalized morphea, glomerulonephritis, glomerulosclerosis, pulmonary fibrosis, glycogenosis, Graft versus host disease, hepatitis B virus infection, hepatitis C virus infection, hepatitis D virus infection, Hypersensitive pneumonitis, hypersensitivity pneumonitides, Hypertensive nephrosclerosis, idiopathic pulmonary fibrosis, Infectious myocarditis, Infectious pneumonitis, inflammatory bowel disease, Inherited disorders, Inherited metabolic disorders, interstitial fibrotic lung disease, interstitial lung disease, Intestinal bypass, Keloid, kidney fibrosis including glomerular sclerosis, kidney fibrosis resulting from dialysis, linear scleroderma, liver cirrhosis, liver fibrosis, lymph node fibrosis, Macular degeneration, mediastinal fibrosis, multifocal fibrosclerosis, myelodysplastic syndrome, myelofibrosis, myeloproferative syndrome, myocardial fibrosis, myocardial infarction, NASH associated cirrhosis obesity, nephrogenic systemic fibrosis, nephropathy, nodular fascilitis, non-alcoholic steatohepatitis (NASH), non-cirrhotic hepatic fibrosis, Nonalcoholic fatty liver disease, oral fibrosis, organ specific fibrosis, pancreatitis, peritoneal fibrosis, Peyronie's disease, Pneumoconiosis, Post-radiation, Pressure-overload heart, primary biliary cirrhosis, progressive kidney disease, Progressive massive fibrosis, progressive renal disease or diabetic nephropathy, protein malnutrition, pulmonary fibrosis, pulmonary fibrosis caused by an infectious agent, pulmonary fibrosis caused by inhalation of inorganic dust, pulmonary fibrosis caused by inhalation of noxious gases, pulmonary hypertension, radiation induced fibrosis, renal fibrosis, renal tubulointerstitial fibrosis, retinal fibrosis, retroperitoneal fibrosis, Sarcoidosis, scarring fibrosis, Schistosomiasis, Scleroderma including morphea, scleroderma/systemic sclerosis, sclerodermatous graft-vs-host-disease, Secondary amyloidosis, silicosis, Storage disorders, e.g. hemochromatosis, Subretinal fibrosis, systemic sclerosis, Toxic environmental exposure, Transplant rejection, trauma induced fibrosis, Tuberculosis, valvular disease, viral induced hepatic cirrhosis, wound healing fibrosis or combinations thereof.

Examples of angiogenesis disorders to where methods to inhibit angiogenesis are needed, include, but are not limited to, cancers (e.g., lung cancer, adenocarcinoma, adenocarcinoma of the lung, squamous carcinoma, squamous carcinoma of the lung, malignant mixed mullerian tumor, head and/or neck cancer, breast cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, stomach cancer, prostate cancer, testicular cancer, ovarian cancer, cervical cancer, endometrial cancer, uterine cancer, pancreatic cancer, colon cancer, rectal cancer, colorectal, gastric cancer, kidney cancer, bladder cancer, spleen cancer, thymus cancer, thyroid cancer, brain cancer, neuronal cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, neuroblastoma, white blood cell cancer (e.g., lymphoma, leukemia, etc.), hereditary non-polyposis cancer (HNPC), and/or colitis-associated cancer), diabetic retinopathy, autoimmune diseases, rheumatoid arthritis, atherosclerosis, cerebral ischemia, neovascular macular degeneration, corneal neovascularization, iris neovascularization, subretinal neovascularization, choroidal neovascularization, cardiovascular diseases or delayed wound healing. In one embodiment, the angiogenesis is not ocular angiogenesis. In one embodiment, the angiogenesis is not corneal neovascularization (CNV).

The method provided herein comprise the administration of siRNA, ASO (Antisense oligonucleotides) or small molecule inhibitors of human FTO, including, but not limited to,

siRNA Sequences of Human FTO

siRNA-1
(SEQ ID NO: 1)
5′-P-GAU CUG CUC ACU CCG GUA UCU-3′
(SEQ ID NO: 2)
5′-P-AGA UAC CGG AGU GAG CAG AUC-3′
siRNA-2
(SEQ ID NO: 3)
5′-P-GAC CUU CCU CAA GCU CAA UGA-3′
(SEQ ID NO: 4)
5′-P-AGA UAC CGG AGU GAG CAG AUC-3′
siRNA-3
(SEQ ID NO: 5)
5′-P-GGU UUC AAG GCA AUC GAU ACA-3′
(SEQ ID NO: 6)
5′-P-AGA UAC CGG AGU GAG CAG AUC-3′

### ASO Sequence of Human FTO:

(SEQ ID NO: 7)
5′-mAmGmGmAmUATTTCAGCTGmCmCmAmCmU-3′
(SEQ ID NO: 8)
5′-mCmCmAmCmUTCATCTTGTCmCmGmUmUmG-3′
(SEQ ID NO: 9)
5′-mAmCmAmUmGCCAAATATCAmGmGmAmUmC-3′
(SEQ ID NO: 10)
5′-mTmCmAmUmUCCTTTGTTCCmAmCmGmGmG-3′

Further inhibitory sequence can be designed from the known FTO genomic and cDNA sequences.

### Inhibitors of Human FTO:

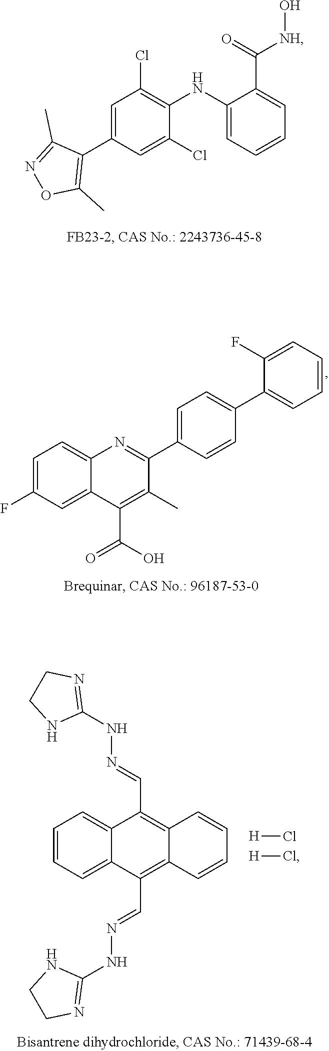

Human Fat mass and obesity-associated protein also known as alpha-ketoglutarate-dependent dioxygenase FTO is an enzyme that in humans is encoded by the FTO gene located on chromosome 16. Its protein sequence can be found at UniProt Accession number Q9C0B1 or accession numbers NP_001073901, NP_001350820, NP_001350823, NP_001350825 or NP_001350826 and cDNA can be found at NM_001080432; CCDS32448.1 (Gene ID: 79068; Chr 16: 53.7-54.16 Mb).

(SEQ ID NO: 11)
MKRTPTAEEREREAKKLRLLEELEDTWLPYLTPKDDEFYQQWQLKYPKLI
LREASSVSEELHKEVQEAFLTLHKHGCLERDLVRIQGKDLLTPVSRILIG
NPGCTYKYLNTRLFTVPWPVKGSNIKHTEAEIAAACETFLKLNDYLQIET
IQALEELAAKEKANEDAVPLCMSADFPRVGMGSSYNGQDEVDIKSRAAYN
VTLLNFMDPQKMPYLKEEPYFGMGKMAVSWHHDENLVDRSAVAVYSYSCE
GPEEESEDDSHLEGRDPDIWHVGFKISWDIETPGLAIPLHQGDCYFMLDD
LNATHQHCVLAGSQPRFSSTHRVAECSTGTLDYILQRCQLALQNVCDDVD
NDDVSLKSFEPAVLKQGEEIHNEVEFEWLRQFWFQGNRYRKCTDWWCQPM
AQLEALWKKMEGVINAVLHEVKREGLPVEQRNEILTAILASLTARQNLRR
EWHARCQSRIARTLPADQKPECRPYWEKDDASMPLPFDLTDIVSELRGQL
LEAKP
(SEQ ID NO: 12)
ATGAAGCGCACCCCGACTGCCGAGGAACGAGAGCGCGAAGCTAAGAAACT
GAGGCTTCTTGAAGAGCTTGAAGACACTTGGCTCCCTTATCTGACCCCCA
AAGATGATGAATTCTATCAGCAGTGGCAGCTGAAATATCCTAAACTAATT
CTCCGAGAAGCCAGCAGTGTATCTGAGGAGCTCCATAAAGAGGTTCAAGA
AGCCTTTCTCACACTGCACAAGCATGGCTGCTTATTTCGGGACCTGGTTA
GGATCCAAGGCAAAGATCTGCTCACTCCGGTATCTCGCATCCTCATTGGT
AATCCAGGCTGCACCTACAAGTACCTGAACACCAGGCTCTTTACGGTCCC
CTGGCCAGTGAAAGGGTCTAATATAAAACACACCGAGGCTGAAATAGCCG
CTGCTTGTGAGACCTTCCTCAAGCTCAATGACTACCTGCAGATAGAAACC
ATCCAGGCTTTGGAAGAACTTGCTGCCAAAGAGAAGGCTAATGAGGATGC
TGTGCCATTGTGTATGTCTGCAGATTTCCCCAGGGTTGGGATGGGTTCAT
CCTACAACGGACAAGATGAAGTGGACATTAAGAGCAGAGCAGCATACAAC
GTAACTTTGCTGAATTTCATGGATCCTCAGAAAATGCCATACCTGAAAGA
GGAACCTTATTTTGGCATGGGGAAAATGGCAGTGAGCTGGCATCATGATG
AAAATCTGGTGGACAGGTCAGCGGTGGCAGTGTACAGTTATAGCTGTGAA
GGCCCTGAAGAGGAAAGTGAGGATGACTCTCATCTCGAAGGCAGGGATCC
TGATATTTGGCATGTTGGTTTTAAGATCTCATGGGACATAGAGACACCTG
GTTTGGCGATACCCCTTCACCAAGGAGACTGCTATTTCATGCTTGATGAT
CTCAATGCCACCCACCAACACTGTGTTTTGGCCGGTTCACAACCTCGGTT
TAGTTCCACCCACCGAGTGGCAGAGTGCTCAACAGGAACCTTGGATTATA
TTTTACAACGCTGTCAGTTGGCTCTGCAGAATGTCTGTGACGATGTGGAC
AATGATGATGTCTCTTTGAAATCCTTTGAGCCTGCAGTTTTGAAACAAGG
AGAAGAAATTCATAATGAGGTCGAGTTTGAGTGGCTGAGGCAGTTTTGGT
TTCAAGGCAATCGATACAGAAAGTGCACTGACTGGTGGTGTCAACCCATG
GCTCAACTGGAAGCACTGTGGAAGAAGATGGAGGGTGTGACAAATGCTGT
GCTTCATGAAGTTAAAAGAGAGGGGCTCCCCGTGGAACAAAGGAATGAAA
TCTTGACTGCCATCCTTGCCTCGCTCACTGCACGCCAGAACCTGAGGAGA
GAATGGCATGCCAGGTGCCAGTCACGAATTGCCCGAACATTACCTGCTGA
TCAGAAGCCAGAATGTCGGCCATACTGGGAAAAGGATGATGCTTCGATGC
CTCTGCCGTTTGACCTCACAGACATCGTTTCAGAACTCAGAGGTCAGCTT
CTGGAAGCAAAACCCTAG

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter is further illustrated by the following specific but non-limiting examples.

### Example

### Materials and Methods

### Animals

C57BL/6 (wild type) mice were purchased from The Jackson Laboratory (Bar Harbor, ME, USA). All mice were housed in a pathogen-free laminar flow facility at the University of Virginia. For all procedures, anesthesia was achieved by intraperitoneal injection of ketamine hydrochloride (100 mg/kg; Fort Dodge Animal Health) and xylazine (10 mg/kg; Phoenix Scientific), and pupils were dilated with topical 1% tropicamide and 2.5% phenylephrine hydrochloride (Alcon Laboratories). Mice were treated in accordance with the guidelines of the University of Virginia's Institutional Animal Care and Use Committee and the Association for Research in Vision and Ophthalmology. Both male and female mice between 6 and 10 weeks of age were used.

### Laser-Induced CNV

Laser photocoagulation (532 nm, 180 mW, 100 ms, 75 μm) (OcuLight GL; IRIDEX Corp., Mountain View, CA, USA) was performed bilaterally (volume studies: 4 spots per eye; RNA analyses: 10 spots per eye) in 6- to 8-week-old mice on day 0 to induce CNV as previously described 1-3. For drug treatment, the mice pretreated with FTO inhibitor (FB23-2, SML2694, Sigma) or vehicle (DMSO) through intraperitoneal injection (i.p, 2 mg/kg). Then, the mice were subjected to laser photocoagulation followed by intravitreous injection of the FTO inhibitor (25 ng/0.5 μl per eye) or vehicle. At day 3 after laser injury, the mice were administered the FTO inhibitor (2 mg/kg) or vehicle via intraperitoneal injection. At day 7, the eyes were collected for fluorescein labeled-isolectin B4 and F4/80 staining and for neovascular volume quantification.

### Cell Culture

Primary WT or mutant mouse bone marrow derived macrophages (BMDMs) were isolated as previously described (4). BMDMs were cultured in IMDM (Gibco) basal medium containing 10% fetal bovine serum (FBS), 30% L929 supernatants, nonessential amino acids, sodium pyruvate, 2-mercaptoethanol, and antibiotics. THP-1 cells were cultured in RPMI 1640 medium with 10% FBS and antibiotics. Human RPE cells were isolated as previously described and cultured in DMEM medium with 10% FBS and antibiotics. All cells were maintained at 37° C. in a 5% CO₂ environment.

### Immunoblotting

Cells and tissues were homogenized in RIPA buffer (R0278, Sigma) with protease and phosphatase inhibitors (A32959, Thermo Scientific). Protein concentration was determined with a Pierce BCA Protein Assay Kit (23225, Thermo Scientific). Equal quantities of protein boiled in LDS Sample Buffer (NP0007, Thermo Scientific) were resolved by Novex Tris-glycine gels (Invitrogen) and transferred onto LF PVDF membranes (1704274, Bio-Rad). The transferred membranes were blocked with Blocking Buffer (927-40000, LI-COR) and incubated with the following primary antibodies: anti-FTO pAb (AdipoGen Life Sciences, AG-25A-0089-C100, USA), anti-GAPDH mAb (Cell Signaling Technology, 97166), anti-F4/80 (Santa Cruz Biotechnology, sc-25830). The signal was visualized with species-specific secondary antibodies conjugated with IRDye and Odyssey® CLx Imaging System.

### Dot Blotting for RNA 6 mA

Total RNA was isolated with Trizol reagent (Invitrogen) and mRNA was purified by Oligo d(T)25 Magnetic Beads (NEB). The same amount of mRNA in a volume of 1.5 μl was denatured by heating at 72° C. for 5 min, followed by chilling on ice. Then, mRNA was spotted on Amersham Hybond-N nylon membrane (GE Healthcare) and cross-linked with UV irradiation (150 mJ/cm2, Stratalinker). The nylon membrane was blocked with 5% BSA in TBST and the m6A level was detected using the m6A-specific antibody (Synaptic Systems, 202003) and the mouse-HRP secondary antibody. Finally, the same membrane was stained with methylene blue as loading control.

### Quantification of RNA 6 mA

The level of global RNA 6 mA levels in LPS-stimulated or unstimulated BMDMs was quantified by EpiQuik m6A RNA Methylation Quantification Kit (Epigentek Group, Farmingdale, NY) by following the manufacturer's instructions.

### RNA-seq

Total RNAs from LPS-stimulated or unstimulated BMDMs were isolated with TRIzol reagents and quantified with Nanodrop. The mRNA purification, library perpetration and RNA-seq was performed on a BGI-SEQ 500 platform and analyzed by BGI (BGI, Hongkong, China).

### Real-Time Polymerase Chain Reaction (RT-PCR)

### Real-Time PCR

Total RNA was extracted from mouse BMDMs or eye tissues with TRIzol™ Reagent (Thermo Fisher Scientific, 15596018, USA) and quantified by using a NanoDrop 2000 spectrophotometer (Thermo-Fisher Scientific, USA). Reverse transcription was performed using the QuantiTect Reverse Transcription kit (205313, QIAGEN). Target genes were amplified by real-time quantitative polymerase chain reaction (PCR) (Applied Biosystems, 7900 HT Fast Real-Time PCR system) with Power SYBR Green Master Mix. Relative gene expression was determined by the 2^{-ΔΔCt} method, and 18S rRNA or GAPDH (glyceraldehyde-3-phosphate dehydrogenase) was used as an internal control. The primers are listed in the Table below:

| Target name | Forward sequence | Reverse sequence |
|---|---|---|
| Mettl14 | TGCTCCAAACTCAAAACGGAA | ATCATTATGGGGATTTAAGCTCT |
| | (SEQ ID NO: 14) | (SEQ ID NO: 15) |
| Mett13 | ATTGTTGAAAAGTTTCGCTCT | GAACATATTTGCAGGTGTCCA (SEQ |
| | (SEQ ID NO: 16) | ID NO: 17) |
| Rbm15 | AGCACTCTAAGCCTCAACACA | ATTTAAAAGGCAACCGGAGCAT |
| | (SEQ ID NO: 18) | (SEQ ID NO: 19) |
| Fto | ACAGCCTCGGTTTAGTTCCAC | TGCCTCAGCCACTCAAACTCC (SEQ |
| | (SEQ ID NO: 20) | ID NO: 21) |
| Wtap | ACAATCACGTCATAGCTACCAG | TGAGACCACACCTTCACGTT (SEQ ID |
| | (SEQ ID NO: 22) | NO: 23) |
| Alkbh5 | CGCGGTCATCAACGACTACCAG | AAAGCACAGGTTCCGACACCC (SEQ |
| | (SEQ ID NO: 24) | ID NO: 25) |
| Vegfa | CACGACAGAAGGAGAGCAGAAG | GCTACTGCCGTCCGATTGAG (SEQ ID |
| | (SEQ ID NO: 26) | NO: 27) |
| Fgfb | GAAGAGCGACCCACACGTCA | CCAGTTCGTTTCAGTGCCACA (SEQ |
| | (SEQ ID NO: 28) | ID NO: 29) |
| Pdgfa | CCTTCCTGATGGCCTTACGTG | AAAATAACCGTCTCGCTGT (SEQ ID |
| | (SEQ ID NO: 30) | NO: 31) |
| Plgf | CTAGAACCTGCCCTGATTCCC | AACCCAGACTTGTATCGGTCA (SEQ |
| | (SEQ ID NO: 32) | ID NO: 33) |
| Gapdh | CAGGTTGTCTCCTGCGACTT (SEQ | TATGGGGGTCTGGGATGGAA (SEQ |
| | ID NO: 34) | ID NO: 35) |
| 18S | TTCGTATTGCGCCGCTAGA (SEQ | CTTTCGCTCTGGTCCGTCTT (SEQ ID |
| | ID NO: 36) | NO: 37) |

### RPE Flat Mounts and Immunofluorescent Staining

RPE flat mounts were prepared as previously described 5. Briefly, at day 3 or 7 after laser injury, mice eyes were enucleated and dissected as retina and sclera-RPE-choroid complexes. The RPE-choroid sheets were immediately fixed in 4% paraformaldehyde (Electron Microscopy Sciences, 50-980-495, USA) in phosphate-buffered saline for 1 hour. Next, the RPE-choroid sheets were permeabilized and blocked with blocking buffer (3% of Normal goat serum, 0.1% Triton-X 100 in PBA). As described previously¹, CNV volumes were determined by labeling with fluorescein labeled Griffonia Simplicifolia Lectin I (GSL I) isolectin B4 (Vector Laboratories, FL-1201, USA). RPE-conjugated F4/80 antibody (Bio-Rad, MCA497PET, USA) immunolabeling was used to identify infiltrating macrophages. FTO expression was visualized by immunolabeling with anti-FTO antibody (AdipoGen Life Sciences, AG-20A-0083, USA), and the specificity of labeling was assessed using isotype serum immunolabeling. The sclera-choroid/RPE complexes were flat mounted with ProLong® Gold Antifade Mountant with DAPI (Fisher Scientific, P36935, USA). Cryo-sections of 4% Paraformaldehyde (Electron Microscopy Sciences, 15714S, USA) fixed eyes were immune-labeled with anti-FTO pAb (AdipoGen Life Sciences, AG-25A-0089-C100, USA), anti-F4/80 (Bio-Rad, MCA497RT, USA) and DAPI. Fluorescence images were captured by Nikon AIR laser scanning confocal microscope.

### Enzyme-Linked Immunosorbent Assay (ELISA)

Secreted IL-6 in the BMDMs medium or serum IL-6, TNF-alpha, and IL-1alpha were detected by ELISA (Mouse IL-1 alpha/IL-1F1 DuoSet, R&D Systems, DY400; Mouse IL-6 DuoSet, R&D Systems, DY406; Mouse TNF-alpha DuoSet, R&D Systems, DY410) according to the manufacturer's instructions.

### VEGFA ELISA

The RPE-choroid complex was isolated from mouse eyes on day 3 after laser injury and treatment with FTO inhibitor or vehicle. Eye tissues were lysed by sonication with immunoprecipitation assay (RIPA) buffer (Sigma-Aldrich, R0278, USA) containing protease inhibitor (Sigma-Aldrich, 11836170001, USA), on ice for 15 minutes. The lysate was centrifuged at 20,000×g for 15 minutes at 4° C., and total protein in the lysates was quantified using the Thermo Scientific™ Pierce™ BCA™ Protein Assay (Fisher Scientific, PI23225, USA). VEGFA protein levels in the supernatants were measured by VEGF DuoSet ELISA (R&D Systems, DY493, USA), following the manufacturer's instructions. Mouse BMDMs or human THP-1 cells (5×10⁵ cells per well) were plated into six-well plates in complete medium and allowed to adhere overnight. Cells were then incubated for 24 hours in the absence or presence of various doses of FTO inhibitor or METTL3 inhibitors (STM2457, Selleck Chemicals). BMDMs were incubated with 125 ng/ml of LPS from Escherichia coli O111:B4 (Sigma-Aldrich, L2630, USA) for 4 hours, followed by FTO inhibitor treatment as mentioned above. The siRNAs (siFto: 5′-P.G.A.G.G.A.U.C.C.A.A.G.G.C.A.A.A.G.A.U.dT.dT 3′ (SEQ ID NO: 38); siCtrl: 5′-P.U.A.A.G.G.C.U.A.U.G.A.A.G.A.G.A.U.dT.dT 3′; SEQ ID NO: 39), were designed and synthesized by GE Healthcare Dharmacon, were transfected twice with BMDMs by using DharmaFECT 4 Transfection Reagent (Dharmacon, T-2004, USA). VEGFA levels in the supernatants were measured by mouse or human VEGF DuoSet ELISA (R&D Systems, DY493 or DY293B, USA), following the manufacturer's instructions.

mRNA Stability Assay

Macrophage Vegfa mRNA stability was determined by using Actinomycin D-induced transcription inhibition as previously described⁶. Briefly, 1×10⁵ BMDM cells per well were seeded in a 12-well plate. After adhering to culture well, the BMDMs were pretreated with DMSO or 5 μM FB23-2 (SML2694, Sigma, USA) to prevent the FTO-mediated mRNA demethylation. After 24 hours, the first well of BMDM were collected as first-time point (t=0 hour) using a cell scraper (NC1890482, Fisher Scientific, USA). The remaining wells were treated with Actinomycin D (10 μg/ml, A9415, Sigma, USA) to inhibit transcription, and cells collected at 1, 2, 4, and 6 hours after Actinomycin D inhibition. The collected cell pellets were subjected to RNA extraction using TRIzol reagent. The Vegfa mRNA was quantified by RT-PCR as described above.

### Lentivirus Production and Transduction

The lentivirus plasmid coding GFP or human FTO were constructed by Vectotbuilder. Lentiviral particles were produced with packaging mix (Abm, LV003) and Lenti-X 293T cells as per the manufacturer's instructions. Lentiviruses were collected 48 h and 96 h post transfection, purified and concentrated with Amicon Ultra centrifugal filters (mwco 100 kDa) and stored at −80° C. ARPE-19 cells were transduced with lentivirus particles mixed with 3 μg/ml polybrene, and positive cell clones were selected by GFP signal and puromycin selections.

### Methylated RNA Immunoprecipitation-qPCR (MeRIP-qPCR)

To validate m6A methylation of Vegfa mRNA and perturbations induced by FTOi treatment, we performed MeRIP-qPCR by following the EpiMark® N6-Methyladenosine Enrichment Kit protocol (New England Biolabs, E1610S, USA). Briefly, total RNA was extracted from mouse BMDMs treated with 5 μM of FTO inhibitor or vehicle for 24 hours by using TRIzol™ Reagent (Thermo Fisher Scientific, 15596018, USA). 100 μg of total RNA from each group was mixed with unmodified RNA (Negative control, NC). Next, 20 μg of these mixtures was incubated with m6A antibody (Cell Signaling Technology, D9D9W, USA) and then pulled down using Pierce™ Protein A/G Magnetic Beads (Thermo Fisher Scientific, 88802, USA) overnight. The enriched m6A positive RNAs were then eluted and purified by NucleoSpin RNA Clean-Up (Macherey-Nagel, 740948.25, USA), and the amounts of m6A-modified Vegfa mRNA or negative control RNAs were determined by reverse transcription and qPCR.

### Animal Model of Septic Shock

To induced septic shock model, male or female C57BL/6 mice (6 to 10 weeks old) were primed with 10 mg/kg poly(I:C) via intraperitoneal injection. 6 hours later, primed mice were injected with 10 mg/kg LPS and monitored three time daily for a total 5 days. For FTO inhibitor treatment, Wild type C57BL/6J mice were pretreated with 20 mg/kg FB23-2 for 16 hours before sepsis induction. Next day, 20 mg/kg FB23-2 together with 10 mg/kg poly(I:C) were intraperitoneally injected, following with LPS challenge at 6 hours after poly(I:C) priming.

### Animal Model of Choroidal Neovascularization (CNV)

Laser photocoagulation (532 nm, 180 mW, 100 ms, 75 μm) (OcuLight GL; IRIDEX Corp., Mountain View, CA, USA) was performed bilaterally (volume studies: four spots per eye; protein analyses: 20 spots per eye) in 6- to 8-week-old male mice on day 0 to induce CNV in a masked fashion. 1 week after the laser injury, eyes were enucleated and fixed with 4% paraformaldehyde for 30 minutes at 4° C. Eyecups were obtained by removing the anterior segments and were washed in PBS, followed by dehydration and rehydration through a methanol series. After blocking in PBS with 1% bovine serum albumin (Sigma-Aldrich Corp., St. Louis, MO, USA) and 0.5% Triton X-100 (Sigma-Aldrich Corp.) for 1 hour at room temperature, eyecups were incubated with 0.7% FITC-isolectin B4 (Vector Laboratories, Burlingame, CA, USA) overnight at 4° C. After washing in PBS with 0.1% Triton X-100, the neurosensory retina was gently detached and severed from the optic nerve. Four relaxing radial incisions were made, and the remaining RPE-choroid-sclera complex was flat mounted in antifade medium (Immu-Mount Vectashield Mounting Medium; Vector Laboratories) and cover slipped. Choroidal neovascularization was visualized using a blue argon laser wavelength (488 nm) and a scanning laser confocal microscope and quantified using Image J software.

### Statistics

Statistical analyses were performed using GraphPad Prism 8.0. Data were expressed as mean±SEM and were analyzed with unpaired two-tailed t test, one-way analysis of variance (ANOVA) with Dunnett's multiple comparisons, or two-way ANOVA with Sidak's multiple comparisons tests. P<0.05 was deemed statistically significant.

### BIBLIOGRAPHY

1 Nozaki, M. et al. Drusen complement components C3a and C5a promote choroidal neovascularization. Proc Natl Acad Sci USA 103, 2328-2333, doi:10.1073/pnas.0408835103 (2006).
2 Sakurai, E., Anand, A., Ambati, B. K., van Rooijen, N. & Ambati, J. Macrophage depletion inhibits experimental choroidal neovascularization. Invest Ophthalmol Vis Sci 44, 3578-3585, doi:10.1167/iovs.03-0097 (2003).
3 Kleinman, M. E. et al. Sequence- and target-independent angiogenesis suppression by siRNA via TLR3. Nature 452, 591-597, doi:10.1038/nature06765 (2008).
4 Weischenfeldt, J. & Porse, B. Bone Marrow-Derived Macrophages (BMM): Isolation and Applications. CSH Protoc 2008, pdb prot5080, doi:10.1101/pdb.prot5080 (2008).
5 Campos, M., Amaral, J., Becerra, S. P. & Fariss, R. N. A novel imaging technique for experimental choroidal neovascularization. Invest Ophthalmol Vis Sci 47, 5163-5170, doi:10.1167/iovs.06-0156 (2006).
6 Ratnadiwakara, M. & Anko, M. L. mRNA Stability Assay Using transcription inhibition by Actinomycin D in Mouse Pluripotent Stem Cells. Bio Protoc 8, e3072, doi:10.21769/BioProtoc.3072 (2018).

### Results/Discussion

Vascular endothelial growth factor-A (VEGFA, also known as VEGF) is an angiogenic factor that regulates the physiological and pathological blood vessel growth (1). Increased abundance of VEGF in the eye underlies many forms of aberrant ocular angiogenesis and resultant vision loss, including in neovascular age-related macular degeneration (nvAMD), proliferative diabetic retinopathy (PDR), ischemic retinal vein occlusion, and retinopathy of prematurity (ROP) (2). Multiple VEGF inhibitors are approved for such ocular angiogenic diseases. Despite the initial, and often dramatic, efficacy of anti-VEGF therapy, real-world and long-term studies are more sobering (3,4). Thus, enhanced understanding about the regulation of ocular VEGF will elucidate the underlying pathological mechanisms and aid in developing new therapeutic strategies.

N6-methyladenosine (m6A), the most abundant post-transcriptional modification of eukaryotic mRNA, plays fundamental roles in regulating biological processes and diseases (5). The m6A modification is dynamic, being “written” by methyltransferase complex components, and “erased” by demethylases, including Fat mass and Obesity-associated protein (FTO) and AlkB Homolog 5, RNA Demethylase (ALKBH5). The best studied effect of the m6A modification is promotion of mRNA instability, thereby affecting target mRNA transcript abundance. Accordingly, m6A RNA modifications are essential for macrophage activation (6), which was found to be crucial for the development of experimental nvAMD in our prior studies (7). However, whether m6A modification of macrophage genes plays a role in nvAMD is unknown.

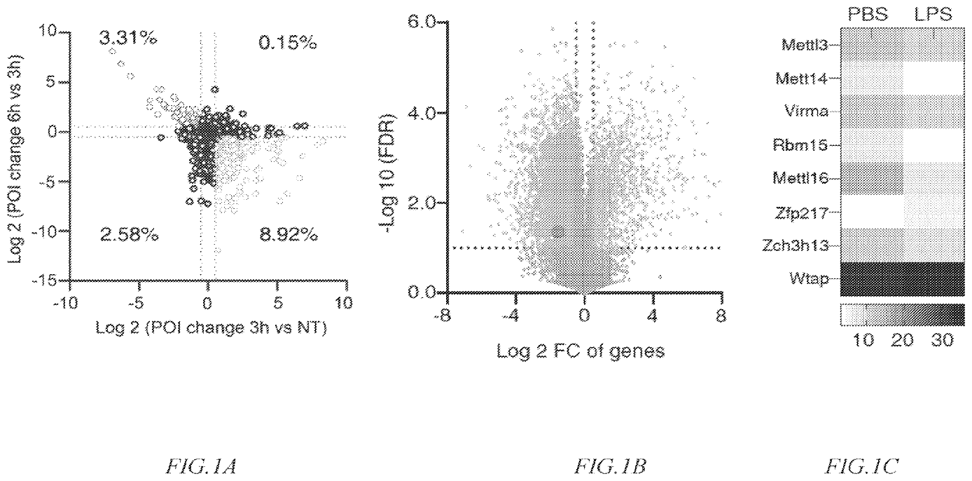

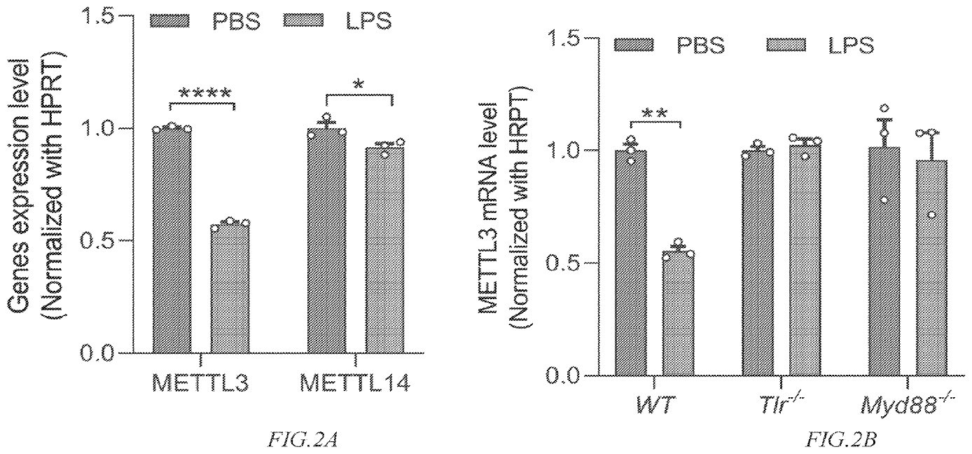

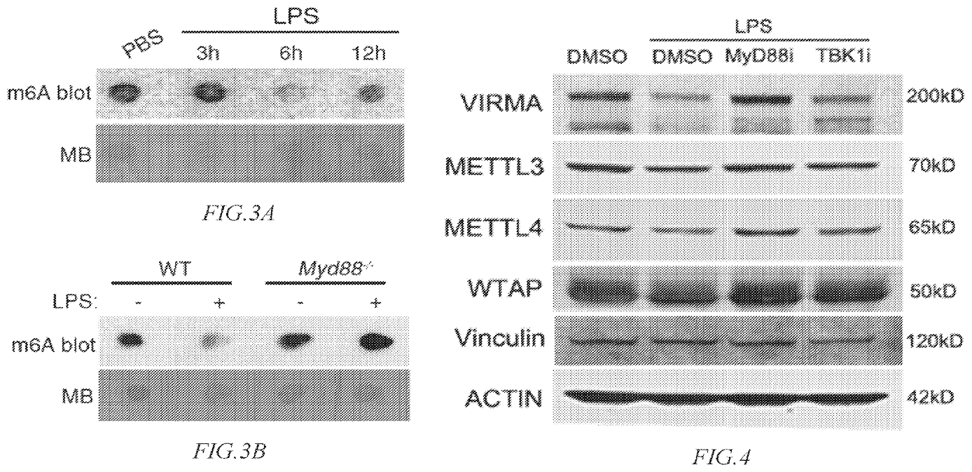

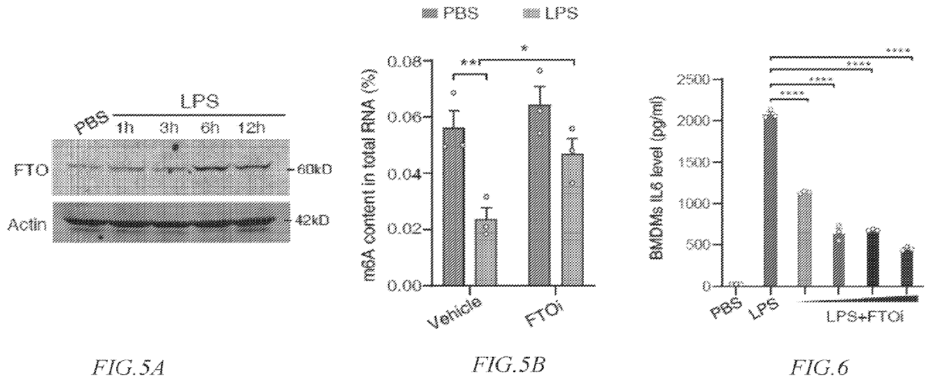

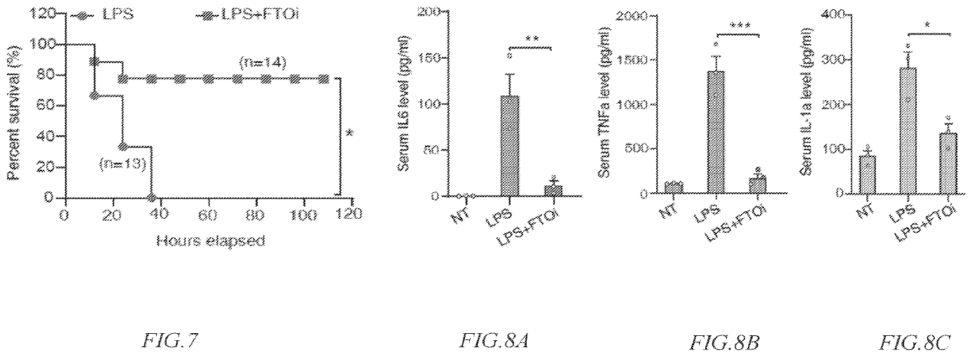

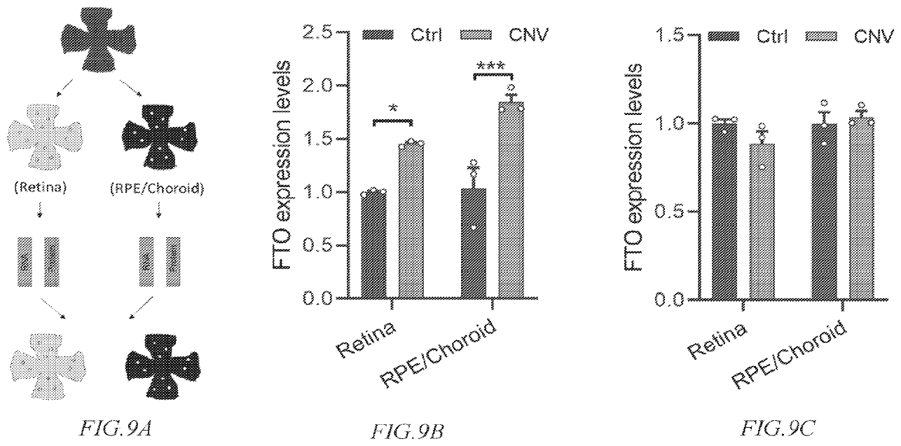

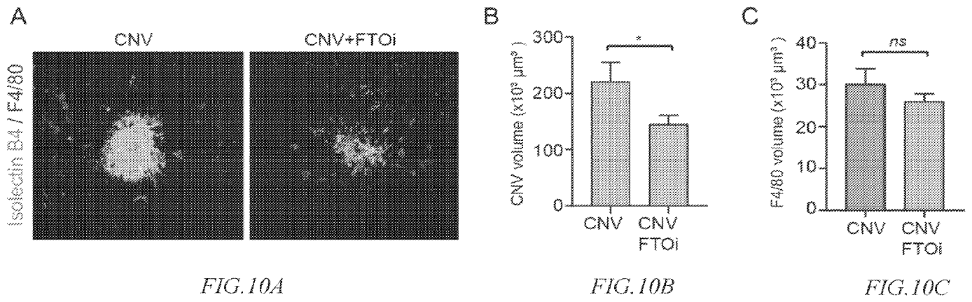

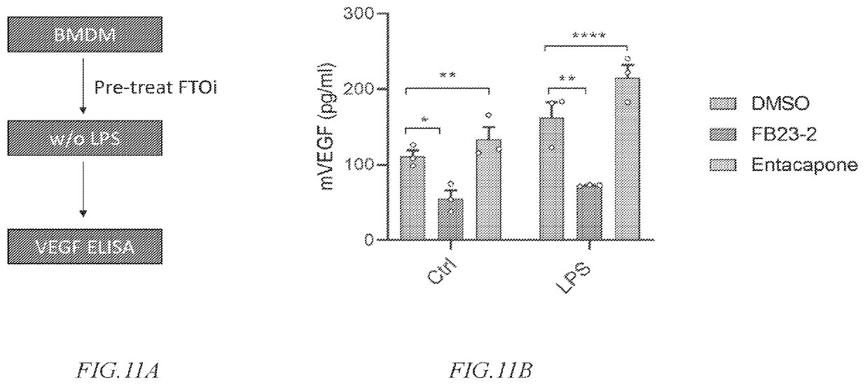

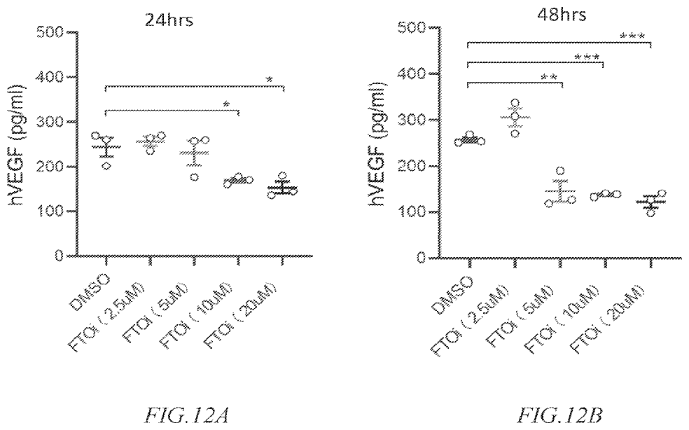

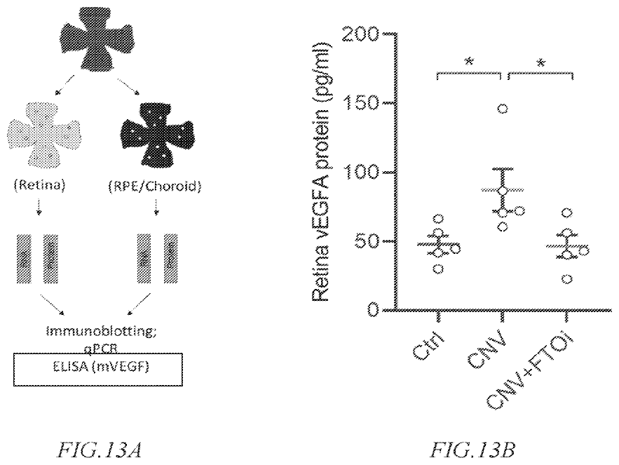

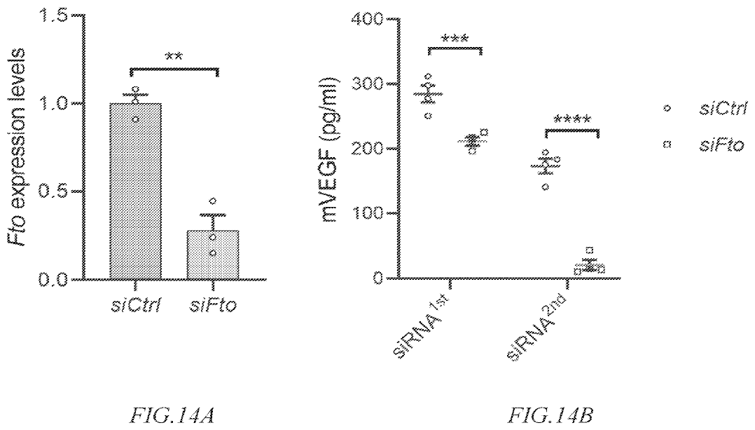

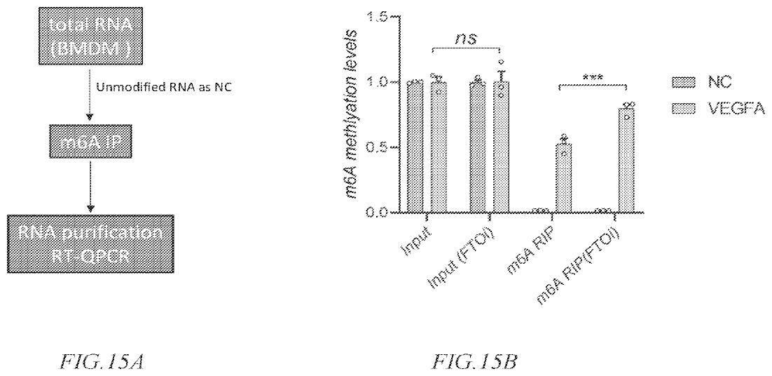

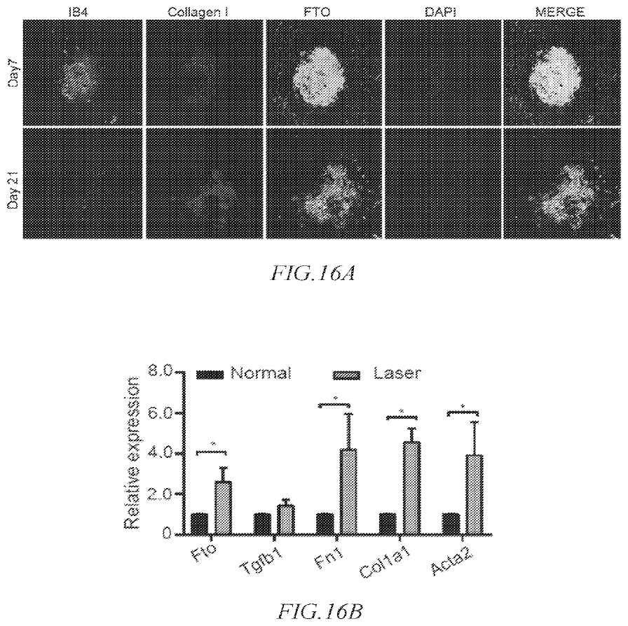

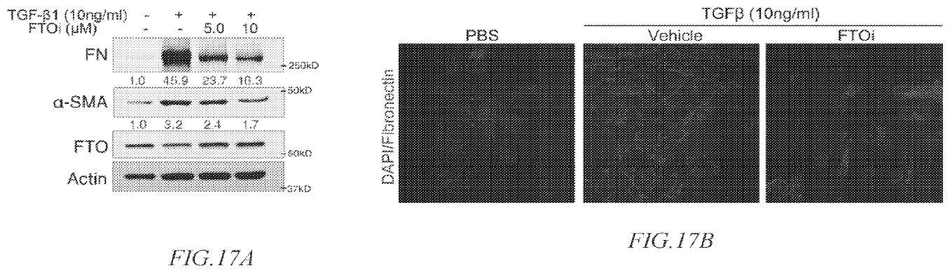

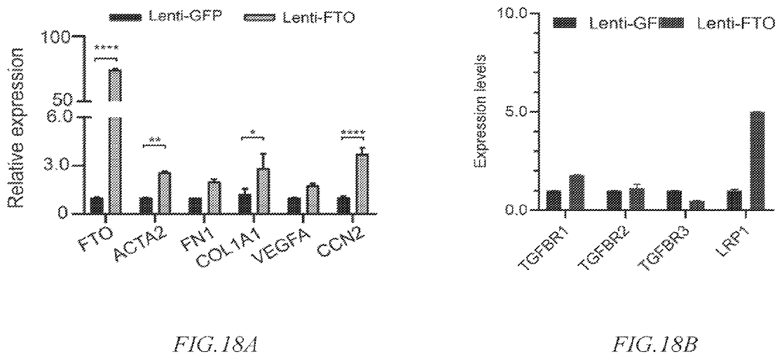

Provided herein in the investigation of role of the m6A methyltranscriptome in laser photocoagulation-induced choroidal neovascularization. An increased abundance of Vegfa mRNA was observed in angiogenic choroid, accompanied by enhanced levels of Fto mRNA and modestly decreased levels of Rbm15 and Wtap mRNAs. However, there were no significant changes in Mettt13, Mettl14, and Alkbh5 mRNA abundance. At 3 days after laser injury, coinciding with macrophage infiltration and the onset of neovascularization (8), a dramatic increase in FTO-expressing cells within the area of neovascularization was observed, some but not all of which were F4/80⁺ (FIG. 1 b, c, d, e and Supplementary FIG. S1b and S1c). Inhibiting FTO activity in vivo using a selective inhibitor resulted in a significant reduction in neovascularization but, interestingly, not in F4/80 macrophage recruitment. In addition, inhibition of FTO suppressed VEGFA protein levels in laser treated RPE-choroid tissue and suppressed the VEGFA release in human ARPE-19 cells. In primary mouse bone marrow derived macrophages, knockdown of FTO expression by specific small interfering RNA (siRNA) significantly dampened macrophage mediated VEGFA release.

Next, Methylated RNA ImmunoPrecipitation PCR (MeRIP-PCR) was used to determine whether FTO demethylated macrophage Vegfa mRNA. Consistent with previous studies (9), we found Vegfa mRNA is abundantly methylated in basal conditions. Inhibition of FTO significantly increased the abundance of m6A methylated Vegfa mRNA; this was accompanied by a reduction in mouse VEGFA release. In contrast, targeting m6A methylase with a METTL3 inhibitor resulted in a dose-dependent increase in VEGFA release. FTO regulates gene expression via maintaining mRNA stability (10). After blocking new mRNA synthesis, we found that macrophage Vegfa mRNA half-life was significantly shorter in the presence of an FTO inhibitor. However, FTO inhibition did not significantly alter the mRNA abundance of other pro-angiogenic factors, such as placental growth factor (Plgf) and platelet-derived growth factors (Pdgfa), suggesting the preferential effect of FTO-mediated m6A demethylation on maintaining macrophage Vegfa mRNA stability and VEGFA release.

Although Vegfa mRNA was the principal pro-angiogenic RNA substrate regulated by FTO in murine macrophages, other genes could also be impacted by FTO inhibition during neovascularization. For example, FTO regulates focal adhesion kinase (FAK) expression in corneal neovascularization (11). The study suggests FTO inhibition has minimal adverse effects on cell viability, and we did not observe in vivo retinal toxicity with FTO inhibition in the studies. However, more detailed toxicity studies remain to be performed. Additionally, macrophage-targeted delivery systems are a promising approach for targeting FTO in ocular angiogenic disorders.

Collectively, the study identifies a previously undescribed role of FTO regulation of VEGFA expression and choroidal neovascularization in vivo (FIG. 11). This work reveals a new mechanism of Vegfa mRNA modification that is regulated by the m6A methyltranscriptome. The discovery that inhibition of FTO suppresses VEGFA release and choroidal neovascularization opens the possibility of therapeutic targeting of FTO for angiogenic eye diseases.

### BIBLIOGRAPHY

1 Shibuya, M. Vascular Endothelial Growth Factor (VEGF) and Its Receptor (VEGFR) Signaling in Angiogenesis: A Crucial Target for Anti- and Pro-Angiogenic Therapies. Genes Cancer 2, 1097-1105, doi:10.1177/1947601911423031 (2011).
2 Nishinaka, A. et al. Pathophysiological Role of VEGF on Retinal Edema and Nonperfused Areas in Mouse Eyes With Retinal Vein Occlusion. Invest Ophthalmol Vis Sci 59, 4701-4713, doi:10.1167/iovs.18-23994 (2018).
3 Glassman, A. R. et al. Five-Year Outcomes after Initial Aflibercept, Bevacizumab, or Ranibizumab Treatment for Diabetic Macular Edema (Protocol T Extension Study). Ophthalmology 127, 1201-1210, doi:10.1016/j.ophtha.2020.03.021 (2020).
4 Peto, T. et al. Long-term Retinal Morphology and Functional Associations in Treated Neovascular Age-Related Macular Degeneration: Findings from the Inhibition of VEGF in Age-Related Choroidal Neovascularisation Trial. Ophthalmol Retina, doi:10.1016/j.oret.2022.03.010 (2022).
5 Yue, Y., Liu, J. & He, C. RNA N6-methyladenosine methylation in post-transcriptional gene expression regulation. Genes Dev 29, 1343-1355, doi:10.1101/gad.262766.115 (2015).
6 Tong, J. et al. Pooled CRISPR screening identifies m(6)A as a positive regulator of macrophage activation. Sci Adv 7, doi:10.1126/sciadv.abd4742 (2021).
7 Sakurai, E., Anand, A., Ambati, B. K., van Rooijen, N. & Ambati, J. Macrophage depletion inhibits experimental choroidal neovascularization. Invest Ophthalmol Vis Sci 44, 3578-3585, doi:10.1167/iovs.03-0097 (2003).
8 Yang, Y. et al. Macrophage polarization in experimental and clinical choroidal neovascularization. Sci Rep 6, 30933, doi:10.1038/srep30933 (2016).
9 Yang, Z. et al. RNA N6-methyladenosine reader IGF2BP3 regulates cell cycle and angiogenesis in colon cancer. J Exp Clin Cancer Res 39, 203, doi:10.1186/s13046-020-01714-8 (2020).
10 Zou, L. et al. N6-methyladenosine demethylase FTO suppressed prostate cancer progression by maintaining CLIC4 mRNA stability. Cell Death Discov 8, 184, doi:10.1038/s41420-022-01003-7 (2022).
11 Shan, K. et al. FTO regulates ocular angiogenesis via m(6)A-YTHDF2-dependent mechanism. Exp Eye Res 197, 108107, doi:10.1016/j.exer.2020.108107 (2020).

One of ordinary skill in the art will recognize that additional embodiments or implementations are possible without departing from the teachings of the present disclosure or the scope of the claims which follow. This detailed description, and particularly the specific details of the exemplary embodiments and implementations disclosed herein, is given primarily for clarity of understanding, and no unnecessary limitations are to be understood therefrom, for modifications will become obvious to those skilled in the art upon reading this disclosure and may be made without departing from the scope of the claimed invention.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A method to treat, inhibit and/or prevent fibrosis comprising administering to a subject in need thereof an effective amount of at least one N6-methyladenosine (m6A) demethylase-inhibiting compound.

2. A method to treat, inhibit and/or prevent angiogenesis in an angiogenesis-dependent disease comprising administering to a subject in need thereof an effective amount of at least one N6-methyladenosine (m6A) demethylase-inhibiting compound.

3. (canceled)

4. (canceled)

5. The method of claim 1, wherein the at least one m6A demethylase-inhibiting compound is a fat mass- and obesity-associated protein (FTO) inhibitor.

6. The method of claim 1, wherein the subject is a mammal.

7. The method of claim 6, wherein the mammal is a human.

8. The method of claim 1, wherein the fibrosis comprises one or more of acute fibrosis, acute kidney injury, adhesive capsulitis, aerosols, Alcoholic liver disease, alpha-1-antitrypsin deficiency, Amyloidosis, Arrhythmogenic right ventricular cardiomyopathy, arthrofibrosis, asbestosis, asthma, Atrial Fibrosis, Autoimmune diseases, Autoimmune glomerular diseases, Autoimmune hepatitis, burn induced fibrosis, carbon pneumoconiosis, cardiac fibrosis, catheter placement, chemical dusts, chemotherapy/radiation induced pulmonary fibrosis, chronic obstructive pulmonary disease (COPD), chronic renal failure, chronic renal insufficiency, Chronic viral hepatitis, cirrhosis, coal worker's pneumoconiosis, collagenous colitis, complications from pneumoconiosis, conjunctival fibrosis, corneal fibrosis, coronary artery disease, Crohn's disease, cystic fibrosis, deltoid fibrosis, diabetes, Diabetic glomerulosclerosis, drug induced ergotism, drug reaction and exposure to toxins, Drug-induced, drug-induced interstitial lung disease, drug-induced interstitial lung disease, Drug-induced nephrogenic systemic fibrosis, Dupuytren's contracture, emerging cirrhosis, end stage renal disease or renal failure, endomyocardial fibrosis, eosinophilic fasciitis, Familial hypertrophic cardiomyopathy, familial pulmonary fibrosis, fibromyalgia, fibrosis associated with atherosclerosis, fumes or vapors, Gaucher's disease, general fibrosis syndrome characterized by replacement of normal muscle tissue by fibrous tissue, generalized morphea, glomerulonephritis, glomerulosclerosis, pulmonary fibrosis, glycogenosis, Graft versus host disease, hepatitis B virus infection, hepatitis C virus infection, hepatitis D virus infection, Hypersensitive pneumonitis, hypersensitivity pneumonitides, Hypertensive nephrosclerosis, idiopathic pulmonary fibrosis, Infectious myocarditis, Infectious pneumonitis, inflammatory bowel disease, Inherited disorders, Inherited metabolic disorders, interstitial fibrotic lung disease, interstitial lung disease, Intestinal bypass, Keloid, kidney fibrosis including glomerular sclerosis, kidney fibrosis resulting from dialysis, linear scleroderma, liver cirrhosis, liver fibrosis, lymph node fibrosis, Macular degeneration, mediastinal fibrosis, multifocal fibrosclerosis, myelodysplastic syndrome, myelofibrosis, myeloproferative syndrome, myocardial fibrosis, myocardial infarction, NASH associated cirrhosis obesity, nephrogenic systemic fibrosis, nephropathy, nodular fascilitis, non-alcoholic steatohepatitis (NASH), non-cirrhotic hepatic fibrosis, Nonalcoholic fatty liver disease, oral fibrosis, organ specific fibrosis, pancreatitis, peritoneal fibrosis, Peyronie's disease, Pneumoconiosis, Post-radiation, Pressure-overload heart, primary biliary cirrhosis, progressive kidney disease, Progressive massive fibrosis, progressive renal disease or diabetic nephropathy, protein malnutrition, pulmonary fibrosis, pulmonary fibrosis caused by an infectious agent, pulmonary fibrosis caused by inhalation of inorganic dust, pulmonary fibrosis caused by inhalation of noxious gases, pulmonary hypertension, radiation induced fibrosis, renal fibrosis, renal tubulointerstitial fibrosis, retinal fibrosis, retroperitoneal fibrosis, Sarcoidosis, scarring fibrosis, Schistosomiasis, Scleroderma including morphea, scleroderma/systemic sclerosis, sclerodermatous graft-vs-host-disease, Secondary amyloidosis, silicosis, Storage disorders, e.g. hemochromatosis, Subretinal fibrosis, systemic sclerosis, Toxic environmental exposure, Transplant rejection, trauma induced fibrosis, Tuberculosis, valvular disease, viral induced hepatic cirrhosis, wound healing fibrosis or combinations thereof.

9. The method of claim 2, wherein the angiogenesis occurs in an angiogenesis dependent disease or disorder comprising one or more cancer, diabetic retinopathy, autoimmune diseases, rheumatoid arthritis, atherosclerosis, cerebral ischemia, neovascular macular degeneration, corneal neovascularization, iris neovascularization, subretinal neovascularization, choroidal neovascularization, cardiovascular diseases or delayed wound healing.

10. The method of claim 9, wherein the cancer comprises one or more of lung cancer, adenocarcinoma, adenocarcinoma of the lung, squamous carcinoma, squamous carcinoma of the lung, malignant mixed mullerian tumor, head and/or neck cancer, breast cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, stomach cancer, prostate cancer, testicular cancer, ovarian cancer, cervical cancer, endometrial cancer, uterine cancer, pancreatic cancer, colon cancer, rectal cancer, colorectal, gastric cancer, kidney cancer, bladder cancer, spleen cancer, thymus cancer, thyroid cancer, brain cancer, neuronal cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, neuroblastoma, white blood cell cancer (e.g., lymphoma, leukemia, etc.), hereditary non-polyposis cancer (HNPC), and/or colitis-associated cancer.

11. The method of claim 1, wherein the inhibitor comprises an inhibitory nucleic acid molecule.

12. The method of claim 11, wherein the inhibitory nucleic acid molecule comprises an antisense nucleic acid molecule, a small interfering RNA (siRNA), or a short hairpin RNA (shRNA).

13. The method of claim 11, wherein the inhibitory nucleic acid molecule comprises one or more of 5′-P-GAU CUG CUC ACU CCG GUA UCU-3′ (SEQ ID NO: 1), 5′-P-AGA UAC CGG AGU GAG CAG AUC-3′ (SEQ ID NO: 2), 5′-P-GAC CUU CCU CAA GCU CAA UGA-3′ (SEQ ID NO: 3), 5′-P-AGA UAC CGG AGU GAG CAG AUC-3′ (SEQ ID NO: 4), 5′-P-GGU UUC AAG GCA AUC GAU ACA-3′ (SEQ ID NO: 5), 5′-P-AGA UAC CGG AGU GAG CAG AUC-3′ (SEQ ID NO: 6), 5′-mAmGmGmAmUATTTCAGCTGmCmCmAmCmU-3′ (SEQ ID NO: 7), 5′-mCmCmAmCmUTCATCTTGTCmCmGmUmUmG-3′ (SEQ ID NO: 8), 5′-mAmCmAmUmGCCAAATATCAmGmGmAmUmC-3′ (SEQ ID NO: 9), and/or 5′-mTmCmAmUmUCCTTTGTTCCmAmCmGmGmG-3′ (SEQ ID NO: 10).

14. The method of claim 1, wherein the inhibitor comprises a small molecule.

15. The method of claim 14, wherein the small molecule comprises one or more of [chemical structure]
